(19) United States
(12) Patent Application Publication
(10) Pub. No.: US 2026/0252771 A1
(43) Pub. Date: Aug. 27, 2026

(54) System and Method for Simulating Underfill Flow during Packaging of Chip Assembly

(71) Applicant: CORETECH SYSTEM CO., LTD., Hsinchu County (TW)

(72) Inventors: Li-Hsuan Shen, New Taipei City (TW); Ching-Kai Chou, New Taipei City (TW); Yu-En Liang, Taipei City (TW); Wei-Yu Lin, Hsinchu County (TW); Zi-Hsuan Wei, New Taipei City (TW); Chia-Hsiang Hsu, Hsinchu County (TW)

(73) Assignee: CORETECH SYSTEM CO., LTD., Hsinchu County (TW)

(21) Appl. No.: 19/422,470

(22) Filed: Dec. 17, 2025

Related U.S. Application Data
(60) Provisional application No. 63/761,176, filed on Feb. 21, 2025.

(51) Int. Cl. G06F 30/28; G06F 111/10; G06F 111/20; G06F 113/18

(52) U.S. Cl. CPC G06F 30/28; G06F 2111/10; G06F 2111/20; G06F 2113/18

(57) ABSTRACT

A system and method for simulating underfill flow during packaging of a chip assembly are provided. The method includes importing an assembly configuration, a bump configuration, and dispensing conditions of an underfill material. A simulation zone is defined based on the assembly configuration, and a corresponding simulation mesh is generated. Bump groups and bump density are determined from the bump configuration to model local flow resistance. The system simulates underfill flow using a thin plate model for confined regions and a porous media model for bump-filled regions, coupled with three-dimensional flow modeling in open areas. Void formation is detected based on the simulated flow results. The hybrid-scale modeling approach provides accurate and efficient prediction of underfill behavior, capillary flow dynamics, and void risks in multi-chip assemblies and fine-pitch semiconductor packaging.

### CROSS REFERENCE TO RELATED APPLICATIONS

This application claims the benefit of U.S. Provisional Application No. 63/761,176, filed on Feb. 21, 2025. The content of the application is incorporated herein by reference.

### BACKGROUND OF THE INVENTION

### 1. Field of the Invention

The present disclosure relates to numerical simulation in semiconductor packaging, and more particularly to a hybrid computational method and system for simulating capillary underfill (CUF) behavior in fine-pitch multi-chip assemblies.

### 2. Description of the Prior Art

In advanced semiconductor packaging, multiple dies are often mounted on a shared substrate or interposer through arrays of solder bumps or micro bumps. After die placement, an underfill material is dispensed to occupy the narrow gaps between the die and the substrate, encapsulating the solder joints to enhance mechanical strength, reliability, and thermal cycling performance. The underfill process is driven primarily by capillary forces, and the resulting flow profile is highly sensitive to parameters such as the gap geometry, bump height and pitch, surface wetting properties, dispensing sequence, and rheological characteristics of the resin.

Traditional optimization of underfill processes has relied heavily on empirical experimentation. However, direct observation of underfill flow in multi-die assemblies is difficult because most semiconductor materials are opaque. Techniques such as transparent mock-up substrates and high-speed imaging have been used, but these methods provide limited accuracy and scalability. Experimental evaluation is also costly and time-consuming, making it impractical to analyze the large design space associated with modern 2.5D and 3D chip packages.

Computational fluid dynamics (CFD) has therefore become an essential alternative for studying capillary underfill (CUF) behavior. Conventional three-dimensional CFD solvers that employ equations can accurately reproduce the flow of viscous materials and surface tension effects. However, when applied to fine-pitch packaging with micro bump arrays, such models require extremely dense computational meshes to resolve individual bumps and narrow flow channels. The total element count can reach billions, resulting in very high memory demand and prolonged computation time. As chip sizes increase and bump pitches shrink below 50 μm, full 3D simulations often become impractical, requiring several weeks or even months of computation for a single case. This computational burden severely limits the use of conventional CFD tools for design iteration or process optimization in production environments.

Efforts to simplify the problem have included empirical flow resistance corrections, volume-averaged porous media models, and reduced-order analytical methods. However, these approaches generally sacrifice accuracy or fail to capture the coupled effects of surface tension, bump density, and multi-scale gap transitions (for example, between die-to-substrate and die-to-die regions). As a result, existing simulation techniques cannot efficiently and accurately predict the complex underfill flow behavior observed in heterogeneous multi-chip modules (MCMs).

Accordingly, there remains a need for a simulation method that maintains high predictive fidelity while reducing computational complexity. The present disclosure addresses these limitations through a hybrid numerical approach that integrates a thin-film (2.5D) model for narrow gap regions with a porous-media-based Equivalent Bump Group (EBG) model to represent the flow resistance induced by micro bumps, coupled with a full 3D Navier-Stokes solver in open regions. This hybrid framework enables fast yet accurate prediction of underfill flow dynamics, void formation, and process optimization in fine-pitch and multi-die semiconductor assemblies such as 2.5D and 3D multi-chip modules (MCMs).

### SUMMARY OF THE INVENTION

An embodiment provides a system and method for simulating underfill flow during packaging of a chip assembly. The system includes a processor with memory configured to execute instructions that import chip assembly configuration data, bump configuration data, and dispensing conditions of an underfill material, define one or more simulation zones based on the assembly geometry, generate a corresponding simulation mesh, and determine bump groups and bump density to model local flow resistance. The processor then simulates the flow of the underfill material using a thin plate model for confined gap regions defined by assembly and dispensing parameters, together with a porous media model defined by the bump density, and detects void formation from the simulation results.

In some aspects, the simulation zone may include different regions of the chip assembly such as die-to-substrate areas, die-to-die gaps, or non-die regions, each treated with appropriate flow models and mesh strategies. For die-to-substrate areas, the simulation mesh comprises a single-layer three-dimensional mesh, and the thin plate model employs the Hele-Shaw approximation to solve drag forces and capillary forces in a two-dimensional field, thereby enabling efficient simulation of horizontal underfill flow beneath the chip. For die-to-die gap areas, the simulation mesh comprises a vertically oriented two-dimensional mesh perpendicular to the substrate, and the thin plate model employs a modified Hele-Shaw approximation to solve drag forces, capillary forces, and hysteresis effects that represent corner resistance as the underfill transitions from horizontal to vertical flow. For non-die areas such as overflow or dispensing regions, the simulation mesh comprises a full three-dimensional mesh, and a three-dimensional flow model employs Navier-Stokes equations to capture complex free-surface behavior and three-dimensional flow patterns.

The porous media model computes flow resistance based on the bump density and grouping of bumps, treating regions with high bump density as porous media with increased viscous drag. The hybrid approach integrates reduced-order two-dimensional models in thin-gap zones with full three-dimensional models in open regions, enabling accurate prediction of underfill flow behavior while substantially reducing computational cost compared to conventional full three-dimensional simulation.

In some aspects, the assembly configuration includes data such as die layout on the substrate, substrate geometry, material composition, gap height between dies and substrate, and material properties of each die. The bump configuration may include bump layout, spatial distribution on the substrate, material properties, and geometric characteristics such as bump pitch, diameter, and height. The dispensing condition may include properties such as viscosity, density, surface tension, flow rate, dispensing sequence, dispensing time, and contact angle between the underfill material and solid surfaces of the chip assembly.

The system enables detection of void formation by analyzing the simulated flow field to identify regions where underfill material has not completely filled the intended gap volume, providing critical feedback for process optimization and design-for-manufacturability in advanced semiconductor packaging applications including fine-pitch multi-chip assemblies, 2.5D and 3D integrated packages, and chip-on-substrate configurations.

To the accomplishment of the foregoing and related ends, certain embodiments comprise the features hereinafter fully described and particularly pointed out in the claims. The following description and accompanying drawings set forth in detail certain illustrative aspects of the embodiments. These aspects are indicative, however, of but a few of the various ways in which the principles of the embodiments may be employed, and the present disclosure is intended to include all such aspects and their equivalents. These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 depicts a cross-sectional schematic of a hybrid simulation model of underfill flow in a chip-to-substrate gap according to the embodiments.

FIG. 2 depicts a cross-sectional schematic of a hybrid simulation model of underfill flow in a die-to-die gap region according to the embodiments.

FIG. 3 depicts a diagram of static interfacial force balance at a liquid-solid contact line according to the embodiments.

FIG. 4 depicts a diagram of interfacial force imbalance and flow hesitation in a confined gap according to the embodiments.

FIG. 5 depicts sequential underfill front positions illustrating the gap resistance effect according to the embodiments.

FIG. 6 depicts a graphical user interface for configuring hybrid and EBG zones, contact angles, and bump density according to the embodiments.

FIG. 7A and FIG. 7B depict a hybrid computational model including hybrid and overflow zones according to the embodiments.

FIG. 8 depicts a flow diagram of a method for simulating underfill flow during chip packaging according to the embodiments.

### DETAILED DESCRIPTION

The following detailed description illustrates exemplary embodiments and explains the principles and operation of a hybrid numerical method and system for simulating underfill flow in semiconductor packaging structures. It should be understood that the embodiments described herein are provided for purposes of explanation and not limitation. Modifications and variations may be made without departing from the scope of the invention as defined in the appended claims.

The embodiments disclose a hybrid underfill flow simulation framework that integrates a thin plate model and a porous media model within a unified computational environment. The system enables efficient and accurate prediction of capillary-driven underfill behavior in fine-pitch multi-chip assemblies, including 2.5D and 3D multi-chip modules (MCMs), chip-on-wafer, and chip-on-substrate structures.

The disclosure recognizes that conventional full three-dimensional computational fluid dynamics (CFD) simulations of underfill flow are computationally expensive due to the extremely small gap heights and dense micro bump arrays in advanced packaging designs. To address this limitation, the hybrid solver introduces a multi-scale modeling approach that couples three-dimensional flow regions with reduced-order two-dimensional approximations in thin-gap zones.

The hybrid framework divides the simulation domain into distinct zones based on the geometry of the chip assembly, including a die-to-substrate zone, modeled using a single-layer thin plate mesh governed by the Hele-Shaw approximation, a die-to-die gap zone, modeled using a modified thin plate model incorporating a gap resistance effect; and a non-die overflow zone, modeled using full 3D Navier-Stokes equations.

Within each zone, the solver accounts for the relevant driving and resisting forces, including capillary pressure, viscous drag, and hysteresis effects at geometric corners. In addition, micro bump distributions are represented through an Equivalent Bump Group (EBG) model, which treats local bump arrays as porous media characterized by an equivalent flow resistance derived from bump density and geometry.

The disclosed hybrid approach enables accurate, multi-scale simulation of underfill flow using a drastically reduced number of mesh elements, achieving computation speeds an order of magnitude faster than traditional 3D methods while maintaining high fidelity to experimental results.

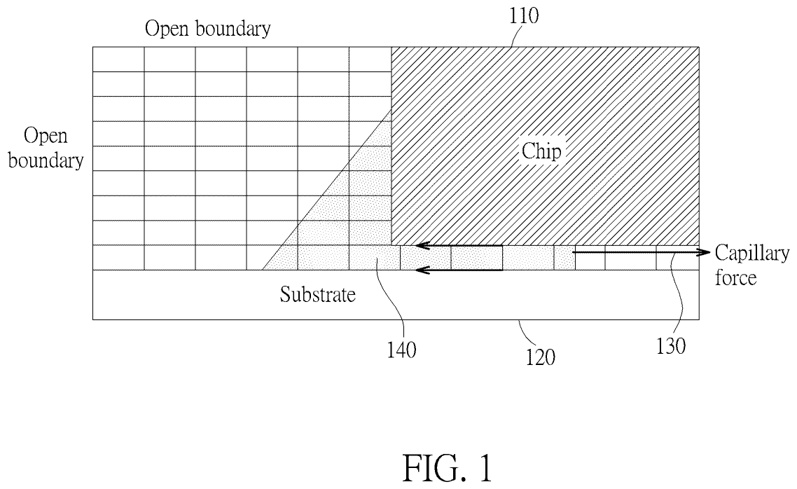

FIG. 1 depicts an exemplary cross-sectional schematic of the fluid flow region analyzed by the hybrid simulation method and system according to the embodiments. The figure illustrates a portion of a semiconductor package in which a chip 110 is mounted on a substrate 120, leaving a narrow gap 130 between the opposing surfaces of the chip and the substrate. During the packaging process, underfill material 140 is dispensed adjacent to the chip 110 and drawn into the gap 130 primarily by capillary forces.

In the embodiment shown, the hybrid solver divides the computational domain into distinct simulation regions. The under-chip region, corresponding to the gap 130, is designated as a thin-gap simulation zone. Within this zone, the flow of the underfill material 140 is governed by a thin plate model derived from the Hele-Shaw approximation, which assumes that the gap thickness is much smaller than its lateral dimensions. This allows the three-dimensional Navier-Stokes equations to be simplified into a two-dimensional form that captures the essential pressure and viscous flow characteristics.

The arrows drag force in FIG. 1 indicate the viscous resistance exerted on the underfill material 140 as it flows between the closely spaced surfaces of the chip 110 and the substrate 120. This drag force arises due to the strong shear gradient within the thin film and is proportional to the viscosity of the underfill material 140 and the inverse of the gap height of the region. The drag force acts opposite to the direction of flow, slowing the advancement of the resin front.

The arrow capillary force represents the surface-tension-driven pressure differential that propels the underfill material 140 into the gap 130. This force is generated by the curvature of the fluid meniscus at the advancing front and the wetting interaction between the underfill material 140 and the solid surfaces of the chip 110 and substrate 120. The magnitude of the capillary force depends on the contact angle, surface energy, and the geometry of the confined channel.

In the illustrated hybrid simulation model, both the drag force and the capillary force are simultaneously considered to compute the dynamic movement of the underfill front. The interplay between these forces governs the resin's penetration rate and determines whether voids or air entrapment may occur beneath the chip 110.

Before running a numerical simulation, the user defines at least one Hybrid Zone in the computational model. The Hybrid Zone specifies the region to be simulated using a 2.5D thin-gap model rather than a full 3D Navier-Stokes model. This definition enables the solver to distinguish which parts of the geometry are sufficiently thin to be efficiently represented by a single-layer mesh. The Hybrid Zone is only activated when the user applies a one-layer mesh configuration, and the solver automatically applies the hybrid-scale formulation incorporating additional drag and driving (capillary) forces. Hybrid Zones are typically defined in die-to-substrate gaps and die-to-die gaps, where the flow field is predominantly planar and the thin film assumption holds true. The remaining regions, such as the open dispensing or overflow areas, continue to be computed with full three-dimensional flow equations.

The mesh region shown beneath the chip 110 in FIG. 1 represents a single-layer finite element mesh employed in the hybrid solver to model the thin-gap region. By reducing the number of mesh layers to one while preserving accurate flow physics through the Hele-Shaw approximation, the simulation achieves a substantial reduction in computational cost compared to full 3D meshing.

The area to the left of the chip 110 indicates an overflow or dispensing region, where the underfill material 140 is initially applied. In this region, the solver utilizes a full three-dimensional Navier-Stokes model to accurately capture the free-surface behavior of the dispensed resin before it enters the confined thin-gap region. Once the resin front reaches the interface between the overflow and thin-gap zones, the solver transitions to the Hele-Shaw-based 2.5D flow model to continue the simulation.

This hybrid domain configuration enables the solver to dynamically couple the 3D flow field in open regions with the 2.5D capillary flow field in the confined regions. The solver computes continuous pressure and velocity fields across these regions, ensuring physical consistency and stable numerical convergence.

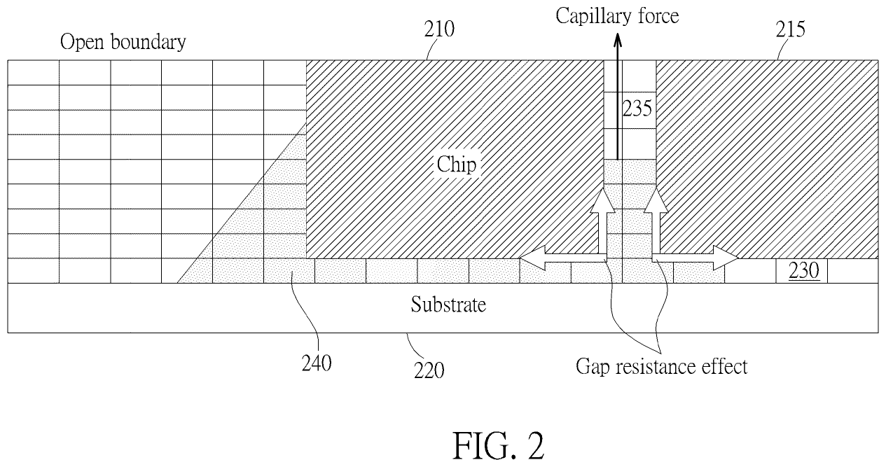

FIG. 2 depicts a cross-sectional schematic of a hybrid simulation model of underfill flow in a die-to-die gap region, according to the embodiments. As illustrated, a first chip 210 and a second chip 215 are mounted on a substrate 220, leaving a narrow vertical gap 230 between the facing edges of the two chips. During the packaging process, an underfill material 240 may be dispensed from one side of the substrate 220 and can flow beneath the chips 210 and 215 by capillary action until it reaches the die-to-die gap 230.

In the embodiment shown, the underfill material 240 can transition from horizontal flow under the chips to vertical filling between the adjacent chips. The horizontal arrows indicate the direction in which the underfill material 240 may flow along the upper surface of the substrate 220 beneath the chips 210 and 215. When the advancing resin front reaches the vertical gap 230, the flow direction may change upward, driven by capillary forces, as indicated by the vertical arrow.

At this transition region, the flow can experience resistance caused by the geometric confinement and the abrupt change in direction. This phenomenon, referred to as the gap resistance effect or corner effect, represents a hesitation or delay in the resin front movement at the corner region between the horizontal and vertical flow paths. The gap resistance effect can occur due to increased viscous drag, localized pressure loss, and complex surface tension behavior within the confined geometry.

In the hybrid simulation model, the gap 230 can be designated as a hybrid zone, which may be numerically modeled using a 2.5D thin-gap model derived from the Hele-Shaw approximation. This model allows the full three-dimensional Navier-Stokes equations to be simplified into a two-dimensional form while retaining critical physical effects such as drag, capillary force, and hysteresis.

The variation of the underfill flow along the vertical direction dominates the behavior in the die-to-die gap region 230. Therefore, instead of constructing a full three-dimensional mesh, the hybrid solver discretizes the gap 230 using a vertically oriented 2D mesh perpendicular to the substrate 220. The solver applies a Hele-Shaw approximation (with possible modifications) on this vertical 2D grid to capture the upward capillary rise of the underfill material 240 as it transitions from the horizontal die-to-substrate region into the vertical die-to-die gap 230. This vertically oriented 2D mesh approach preserves the essential physical effects—including vertical capillary rise, corner hesitation, and gap resistance—while avoiding the computational cost of a full 3D mesh. The 2D grid extends vertically between the facing edges of the first chip 210 and the second chip 215, enabling accurate prediction of the flow behavior with significantly reduced computational resources.

To accurately capture the observed flow hesitation, the hybrid solver can introduce a gap resistance model that adds extra drag and delay terms into the governing equations. These terms may represent the additional resistance encountered by the underfill material 240 as it flows through the narrow corner regions between the first chip 210 and the second chip 215.

The mesh pattern shown beneath the chips 210 and 215 represents a single-layer computational mesh that can be applied to the hybrid zone. This single-layer mesh may enable accurate representation of the underfill flow behavior while substantially reducing the number of computational elements compared to a full 3D mesh, thereby improving simulation efficiency.

The hybrid solver can dynamically couple the thin-gap (2.5D) region with the surrounding three-dimensional flow regions, such as the open dispensing area located to the left of the first chip 210. The solver may ensure continuity of pressure and velocity fields across these regions, enabling a smooth and physically consistent transition between 3D and 2.5D models. Hence, the hybrid simulation method can model capillary-driven underfill flow between the chips 210 and 215 on the substrate 220. The model may effectively capture both the vertical capillary flow in the die-to-die gap 230 and the gap resistance effect at the flow transition corners, thereby providing an accurate and computationally efficient approach for simulating complex underfill behavior in multi-chip assemblies.

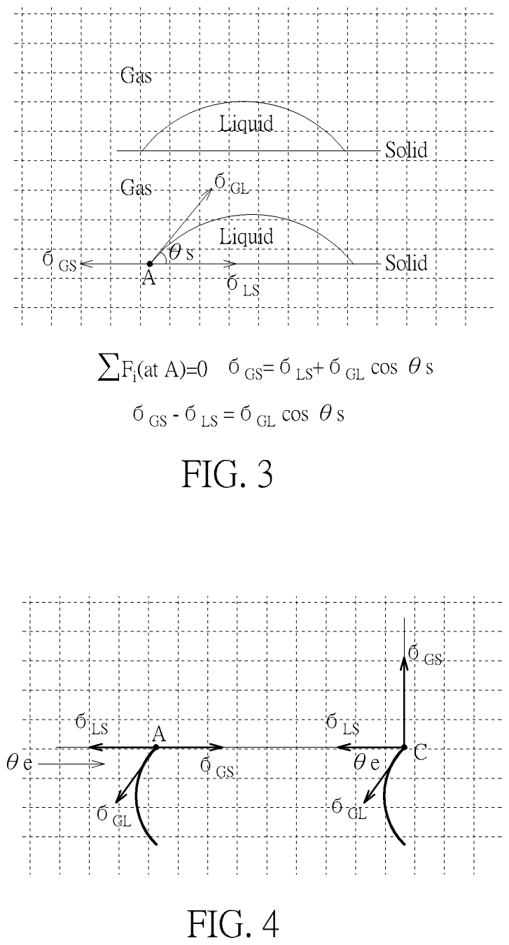

FIG. 3 illustrates a diagram showing the static balance of interfacial forces according to the embodiments. The static balance of interfacial forces acts at the contact line of a liquid droplet on a solid surface. This condition represents the fundamental principle of surface tension equilibrium, which governs the static contact angle (θₛ) of a liquid on a solid substrate.

As illustrated, a liquid phase is positioned between a gas phase and a solid surface. The curved interface between the gas and the liquid represents the meniscus formed due to surface tension. The contact point A marks the intersection of the three phases, i.e., gas, liquid, and solid, where the equilibrium of surface tension forces is established.

At point A, three surface tension vectors act simultaneously. σ_{GS} represents the interfacial tension between the gas and the solid. σ_{LS} represents the interfacial tension between the liquid and the solid. σ_{GL} represents the interfacial tension between the gas and the liquid. The vector σ_{GL} acts along the liquid-gas interface at an angle θₛ relative to the solid surface. The other two vectors, σ_{GS} and σ_{LS}, act tangentially along the solid surface in opposite directions.

In a static or balanced condition, the summation of forces at the contact point A is zero, as expressed by the equilibrium equation:

$$\sum {F}_{i} ( at A ) = 0$$

Accordingly, the balance of interfacial tensions can be written as:

$${σ}_{G S} = {σ}_{L S} + {σ}_{G L} cos {θ}_{s}$$

or equivalently,

$${σ}_{G S} - {σ}_{L S} = {σ}_{G L} cos {θ}_{s}$$

This relationship is known as Young's equation, which defines the static contact angle θₛ as a function of the interfacial energies between the three phases. The static contact angle represents the equilibrium condition where the liquid droplet neither spreads further nor retracts on the solid surface.

In the context of the present disclosure, this static contact angle is a critical parameter used in the simulation of underfill flow. It determines the magnitude of the capillary driving force acting on the underfill material along solid boundaries such as chip and substrate surfaces. Accurate modeling of θₛ enables the hybrid solver to predict the flow front behavior and wetting characteristics of the underfill material under static conditions prior to dynamic flow simulation.

FIG. 4 depicts a diagram showing the interfacial force imbalance according to the embodiments. The interfacial force imbalance can occur at different positions of a liquid front during underfill flow. This figure illustrates two characteristic contact line conditions, point A and point C, used to explain the local variation of surface tension forces when the underfill front encounters geometrical constraints or transitions between surfaces.

The liquid-gas interface is inclined at an angle θₑ, known as the equilibrium contact angle.

At point A, the interfacial forces satisfy the following condition:

$${σ}_{G S} > {σ}_{L S} + {σ}_{G L} cos {θ}_{s}$$

This inequality indicates that a net horizontal component of the surface tension acts to move the liquid front forward, enabling the underfill material to continue spreading along the solid surface. Thus, the front at point A is in an advancing state.

At point C, the local surface geometry changes abruptly, such as when the underfill reaches a vertical wall or a corner. The vector σ_{GS} points upward along the vertical surface, while σ_{GL} and σ_{LS} act horizontally along the lower plane. In this configuration, the capillary driving force in the horizontal direction diminishes significantly. When the equilibrium contact angle θₑ exceeds the static contact angle θₛ, the meniscus becomes too narrow to fully develop, resulting in a reduction of the capillary driving pressure. Consequently, the advancing liquid front loses momentum and may hesitate or stop near the corner.

This phenomenon, referred to as the gap resistance effect, arises from the combined influence of interfacial hysteresis, abrupt geometric transitions, and limited space for meniscus formation. It is a critical factor in confined underfill flow regions, such as die-to-die or die-to-substrate gaps in fine-pitch chip assemblies. The hybrid solver of the present invention incorporates this effect through a gap resistance model, which introduces additional drag and delay terms in the governing equations to reproduce the observed hesitation of the resin front.

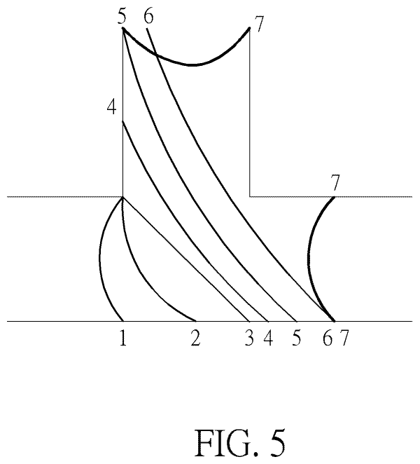

FIG. 5 depicts a diagram showing the evolution of an underfill flow front over time within a confined corner geometry according to the embodiments. This figure demonstrates the liquid front sequentially progresses through the narrow gap region and how local geometric resistance affects its movement, leading to the gap resistance effect.

In the embodiment, the curves 1-7 represent the successive positions of the underfill front as time progresses. The blue lines correspond to the front positions in earlier time steps, while the pink lines indicate later time steps when the front encounters a geometric transition. The flow initially advances along the horizontal surface, following the curves 1 through 4. As the front reaches the vertical wall at position 4, it must change direction to move upward.

The change in flow direction introduces additional viscous resistance and reduces the effective driving force. This resistance is represented by the gap resistance effect, shown as a delay in the upward progression between curves 5 and 7. During this stage, the capillary pressure that drives the underfill into the vertical gap is counteracted by increased drag and contact angle hysteresis, which can cause the front to hesitate or deform before reaching full development.

In narrow geometries, the meniscus curvature and surface wetting condition vary dynamically, and when the equilibrium contact angle θₑ exceeds the static contact angle θₛ, the flow path becomes too narrow for the liquid front to fully develop. This leads to a localized stagnation region or void formation risk, which the hybrid solver should account for.

To capture this phenomenon accurately, the hybrid solver introduces a hesitation term or resistance coefficient in the governing thin-gap model equations. This term quantifies the temporal delay of the advancing resin front as it passes through corner or turning regions of the geometry. The resistance term is activated when the model detects a geometric node corresponding to a die-to-die gap transition or sharp corner.

The sequence of front positions shown in FIG. 5 thus illustrates the time-dependent behavior of the underfill resin under the combined influence of capillary forces, viscous drag, and geometric confinement. Hence, it demonstrates the gap resistance effect manifests as a temporary slowdown of the underfill flow front when transitioning through sharp corners or narrow passages. This physical behavior is modeled in the hybrid solver through additional resistance terms in the 2.5D thin-gap formulation, enabling realistic simulation of flow hesitation and void prediction in advanced multi-chip packaging structures.

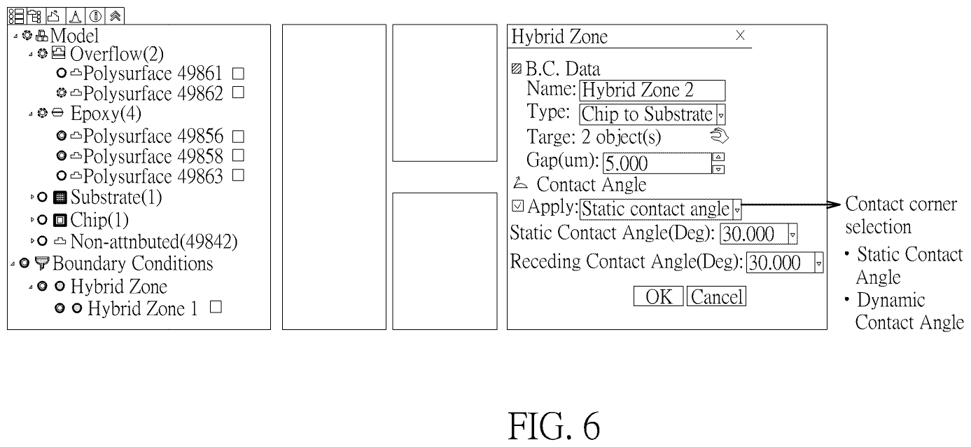

FIG. 6 illustrates an graphical user interface (GUI) for configuring the hybrid simulation according to the embodiments. The interface enables users to define hybrid zones, EBG zones (Encapsulant Between Gaps), and related parameters such as bump density, contact angle, and gap height for accurate simulation of underfill flow behavior in a semiconductor package.

As shown, the model tree on the left side of the interface organizes all geometry and material components of the chip assembly, including overflow, epoxy, substrate, and chip groups. Beneath these, the boundary conditions section lists multiple user-defined hybrid zones, each represented by an orange-highlighted region in the model layout. These hybrid zones correspond to areas where the 2.5D thin-gap solver is activated to simulate underfill flow between closely spaced surfaces, such as chip-to-substrate or die-to-die gaps.

The hybrid zone configuration panel on the right allows the user to specify detailed boundary condition (B.C.) parameters, including the name, type, associated surfaces, and physical gap height. The type field can be set to “chip to substrate” or “die to die,” depending on the physical interface being modeled. The Gap (μm) entry defines the vertical spacing between the corresponding surfaces.

The contact angle section provides control over surface wetting behavior, a key factor influencing capillary-driven underfill flow. Users can apply either a static contact angle or a dynamic contact angle, which includes both advancing and receding components. The static contact angle represents the equilibrium wetting state, while advancing and receding angles are used to simulate hysteresis effects that occur during the motion of the underfill front over different materials or surface textures.

The user can select apply: static contact angle from the dropdown menu, as illustrated, and input numerical values for the angles. In the shown configuration, the static contact angle is set to 30 degrees, which can represent a moderately wetting epoxy material in contact with silicon or substrate surfaces.

In addition to hybrid zones, the system allows definition of an EBG zone. The EBG zone is used to model underfill flow in narrow vertical channels or die-to-die gaps, where the geometry differs from the planar chip-to-substrate region. The EBG zone employs a hybrid computation approach similar to that of the hybrid zone but incorporates additional corrections for corner resistance and vertical capillary rise.

In practice, the EBG zone may be used to represent the narrow spacing between two adjacent dies mounted on the same substrate. The flow behavior in this zone is influenced not only by capillary forces but also by geometric constraints that alter the local pressure distribution. The solver includes terms to account for gap resistance, hysteresis, and partial meniscus formation, ensuring accurate simulation of filling behavior in fine-pitch or stacked-die configurations.

Another critical parameter incorporated into the hybrid solver is bump density, which quantifies the spatial distribution of solder bumps or micro-pillars within the simulation zone. Bump density determines the porous resistance that the underfill experiences as it flows through the bump array.

The solver treats regions with high bump density as porous media, introducing an additional flow resistance term. The bump density is typically computed based on the layout data of the chip assembly, including bump pitch, diameter, and arrangement pattern. The user can import this data as part of the bump configuration file or specify it through the solver's preprocessing interface.

Regions of varying bump density can be automatically grouped into bump clusters or density zones, each with a distinct permeability value. These clusters allow the solver to model heterogeneous flow behavior accurately, for example, slower resin penetration in dense bump regions and faster flow in sparse or open regions.

Both the hybrid zone and EBG zone configurations integrate within the solver environment. The hybrid solver automatically couples these zones with the full 3D flow regions defined elsewhere in the model, maintaining continuous pressure and velocity fields. The contact angle and bump density data directly influence the calculation of capillary forces and viscous drag, enabling physically accurate prediction of underfill advancement, hesitation, and void formation.

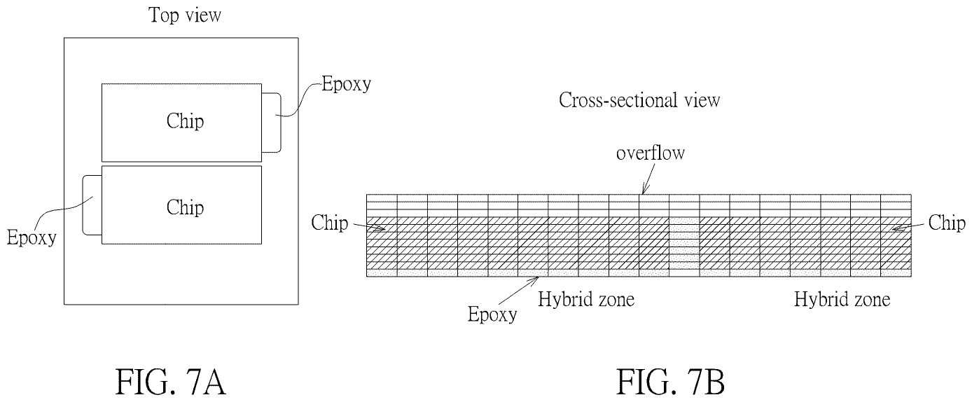

FIGS. 7A and 7B depict the computational model for underfill flow simulation according to the embodiments. Both a top view (FIG. 7A) and a cross-sectional view (FIG. 7B) of a hybrid simulation domain are illustrated. The illustrations include multiple hybrid zones and overflow regions. These figures demonstrate that the hybrid solver partitions the chip assembly geometry into distinct computational regions for efficient and accurate simulation of the underfill filling process.

FIG. 7A presents a top view of a semiconductor package including two chips mounted on a substrate. The two rectangular regions represent the chips, while smaller adjacent rectangular regions indicate overflow zones, where the underfill material is initially dispensed before being drawn into the narrow gaps beneath the chips by capillary action. This top-view layout defines the starting points and directions of the underfill flow. Each chip corresponds to a hybrid zone, which is defined in the simulation setup to represent the thin gap between the chip and the substrate. These zones enable the solver to apply the appropriate flow models in regions with confined geometries.

FIG. 7B presents a cross-sectional view of the same package model, showing the vertical structure of the computational mesh. The lower layer represents the hybrid zone, corresponding to the thin gap region between the chip and the substrate. In this region, the solver applies a thin-gap flow model based on the Hele-Shaw approximation, which simplifies three-dimensional viscous flow to an equivalent two-dimensional form while preserving the effects of drag and capillary forces.

Above the hybrid zone is the epoxy region, which represents the primary underfill flow domain. The overflow region, located near the upper boundary of the cross-sectional view, represents the inlet area where underfill material is dispensed and begins to enter the chip assembly. The solver computes this region using the full three-dimensional Navier-Stokes equations to resolve free-surface behavior before the material enters the confined thin-gap region.

In the embodiment shown, two hybrid zones are defined with one beneath each chip. Each hybrid zone can have independently specified parameters such as gap height, contact angle, and bump density. The gap height defines the thickness of the thin gap to be modeled. The contact angle controls the magnitude of the capillary driving force between the underfill material and the solid surface. The bump density defines the porous resistance to flow caused by the presence of solder bumps or micro-pillars within the gap.

Together, FIGS. 7A and 7B illustrate the integration of both 3D and 2.5D flow models within a single computational framework. The overflow regions are treated with full 3D free-surface flow analysis, while the hybrid zones apply thin-gap modeling to capture capillary-driven flow in confined geometries efficiently. This hybrid approach provides accurate prediction of underfill advancement, hesitation, and void formation, while significantly reducing computational time compared to fully three-dimensional simulation of the entire package.

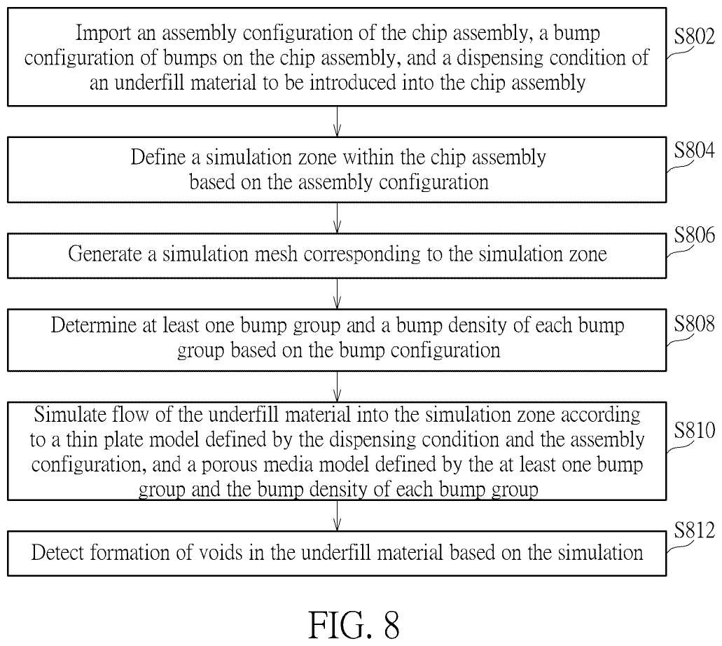

FIG. 8 depicts a flow diagram of a method 800 for simulating underfill flow during packaging of a chip assembly according to the embodiments. The method represents a structured computational sequence executed by the hybrid solver to predict the flow behavior of underfill material and identify potential void formation in a semiconductor package. The method 800 includes the following steps:
S802: Import an assembly configuration of the chip assembly, a bump configuration of bumps on the chip assembly, and a dispensing condition of an underfill material to be introduced into the chip assembly;
S804: Define a simulation zone within the chip assembly based on the assembly configuration;
S806: Generate a simulation mesh corresponding to the simulation zone;
S808: Determine at least one bump group and a bump density of each bump group based on the bump configuration;
S810: Simulate flow of the underfill material into the simulation zone according to a thin plate model defined by the dispensing condition and the assembly configuration, and a porous media model defined by the at least one bump group and the bump density of each bump group; and
S812: Detect formation of voids in the underfill material based on the simulation.

The process begins with step S802, where the system imports all necessary simulation input data, including the assembly configuration, bump configuration, and dispensing conditions. The assembly configuration defines the physical structure of the chip package, including die layout, substrate geometry, material composition, and gap dimensions between dies and substrate. This configuration data may be imported from CAD files, layout databases, or industry-standard formats such as GDSII or OASIS. The bump configuration provides detailed information on bump geometry, spatial distribution, material properties, and bump array patterns. This data includes bump pitch, diameter, height, and arrangement (e.g., area array, peripheral array), and may be imported from package design files or bump map data. The dispensing condition specifies process parameters such as underfill material viscosity, density, surface tension, dispensing sequence, dispensing time, flow rate, and contact angles with various surfaces. These parameters define the fluid properties and process conditions that govern the capillary-driven flow behavior. Together, these data inputs establish the complete geometric, material, and process conditions necessary for accurate simulation.

In step S804, the system analyzes the imported geometry and defines one or more simulation zones based on the assembly configuration. These zones represent distinct regions where the flow characteristics differ due to geometric confinement or material transitions. The system may automatically identify and classify zones such as die-to-substrate areas (narrow horizontal gaps between chip bottom surface and substrate top surface), die-to-die gap areas (narrow vertical gaps between adjacent chip edges), overflow or dispensing regions (open areas where underfill is initially applied), and edge regions (boundaries where flow transitions occur). Each zone is assigned an appropriate modeling approach based on its geometric characteristics. Die-to-substrate zones are designated as horizontal hybrid zones where a thin-gap model with single-layer mesh will be applied. Die-to-die zones are designated as vertical hybrid zones where a vertically oriented 2D mesh will be employed. Overflow zones are designated as full 3D regions where Navier-Stokes equations will be solved. This classification allows the solver to balance computational efficiency and accuracy by applying reduced-order models only where the thin-gap assumption is valid, while maintaining full 3D resolution in regions with complex free-surface behavior.

Next, in step S806, the system generates a computational mesh to discretize each simulation zone. The mesh generation process adapts to the zone type and geometric complexity. For die-to-substrate horizontal hybrid zones, the system creates a single-layer 3D mesh in which the gap region is represented by a thin layer of elements spanning the area beneath the chip. The mesh resolution is determined based on feature size, expected flow gradients, and accuracy requirements. For die-to-die vertical hybrid zones, the system generates a vertically oriented 2D mesh perpendicular to the substrate, with mesh nodes arranged along the vertical gap between adjacent chip edges. This 2D mesh captures the vertical variation of the flow while reducing computational degrees of freedom. For overflow and non-die regions, the system constructs a full 3D tetrahedral or hexahedral mesh with multiple layers to resolve three-dimensional flow patterns and free-surface behavior. The mesh may incorporate refinement near geometric features such as chip corners, dispensing points, or regions with high flow gradients. Transition regions between different zone types are meshed with compatible node arrangements to ensure continuity of pressure and velocity fields. The resulting mesh structure enables the solver to compute flow variables accurately across the entire domain while minimizing the total element count through zone-specific mesh strategies.

In step S808, the solver processes the bump configuration data to determine bump grouping and local bump density throughout the simulation zones. The bump density quantifies the proportion of solid area occupied by solder bumps or micro-pillars within a given region, and serves as the basis for computing local flow resistance. The system may employ automated algorithms to analyze the bump layout and identify clusters or groups of bumps with similar spatial density. Regions with high bump density are characterized by closely spaced bumps that create significant obstruction to underfill flow, resulting in increased viscous drag and reduced penetration rate. Conversely, regions with lower bump density or sparse bump distribution permit faster underfill advancement. Each identified bump group is assigned a characteristic density value, typically expressed as a volume fraction or area fraction of solid material. The system then computes an equivalent permeability or flow resistance coefficient for each bump group based on porous media theory. This coefficient represents the drag force per unit velocity exerted by the bump array on the flowing underfill. The permeability may be calculated using empirical correlations, analytical models (such as the Carman-Kozeny equation), or pre-computed lookup tables based on bump geometry and spacing. Regions of varying bump density are mapped onto the simulation mesh, creating a heterogeneous resistance field that accurately represents the spatial variation of flow impedance. This bump grouping and density mapping enable the solver to model the interaction between underfill flow and bump arrays without explicitly meshing each individual bump, thereby achieving a substantial reduction in computational cost while preserving the essential flow physics.

In step S810, the solver simulates the transient flow of underfill material into the defined simulation zones by solving coupled governing equations that incorporate multiple physical models. For die-to-substrate horizontal hybrid zones represented by single-layer meshes, the solver applies a thin plate model derived from the Hele-Shaw approximation. In this model, the three-dimensional Navier-Stokes equations are integrated across the gap height, yielding a two-dimensional pressure-driven flow equation that accounts for viscous drag between the closely spaced chip and substrate surfaces. The driving pressure is determined by capillary forces arising from surface tension and wetting angle, while the resisting forces include viscous drag proportional to fluid viscosity and inversely proportional to gap height, as well as porous resistance from the bump array characterized by the permeability field established in step S808. For die-to-die vertical hybrid zones represented by vertically oriented 2D meshes, the solver applies a modified Hele-Shaw model that captures vertical capillary rise. This model incorporates additional terms to represent the gap resistance effect or corner hesitation that occurs when the underfill transitions from horizontal flow beneath a chip to vertical flow in the narrow gap between adjacent chips. The gap resistance is modeled as a localized drag or delay term activated at geometric corners, representing the increased viscous dissipation and reduced capillary driving force associated with the abrupt change in flow direction and meniscus curvature. For overflow and non-die regions represented by full 3D meshes, the solver applies the complete Navier-Stokes equations coupled with a volume-of-fluid (VOF) or level-set method to track the free surface of the advancing underfill front. These equations resolve the three-dimensional velocity and pressure fields, accounting for gravitational forces, surface tension, dynamic contact angle, and viscous stresses. The solver ensures continuity of flow variables across boundaries between different zone types by imposing appropriate interface conditions that match pressure, velocity, and mass flux. The simulation proceeds in a time-stepping manner, advancing the underfill front incrementally and updating the pressure and velocity fields at each time step. At each step, the solver evaluates the capillary pressure based on the local meniscus curvature and contact angle, computes the viscous drag from both wall shear and porous resistance, and solves for the resulting flow field. The advancement of the resin front is governed by the balance between capillary driving forces and viscous resisting forces, with the local velocity determined by the pressure gradient and flow resistance. This coupled multi-physics simulation captures the complex interplay of surface tension, wetting, viscosity, gap geometry, and bump-induced resistance, enabling accurate prediction of underfill penetration rate, flow path, and filling pattern.

In step S812, the solver analyzes the computed flow field and resin front evolution to detect the formation of voids or air entrapment within the underfill material. Void formation typically occurs in regions where the underfill front does not advance uniformly, resulting in pockets of trapped air that remain unfilled. The void detection algorithm examines the final filled volume and identifies regions within the nominal chip-to-substrate or die-to-die gap that have not been reached by the underfill material at the end of the dispensing process. The system may employ several criteria to identify potential voids, including detecting cells or nodes in the simulation mesh where the underfill volume fraction remains below a threshold value (e.g., less than 95 percent filled), identifying isolated air pockets surrounded by filled regions, which indicate that the underfill front has bypassed or encircled these areas, locating regions where the flow velocity dropped below a critical value and remained stagnant for an extended period, suggesting insufficient capillary driving force to complete filling, and analyzing the resin front morphology to identify cusps, pinch-offs, or bifurcations that indicate non-uniform advancement and potential void nucleation sites. The solver may also evaluate pressure distribution to identify low-pressure zones that are prone to air entrapment. Once potential void locations are identified, the system quantifies the void characteristics, including void volume, void position relative to chip and bump features, void shape and aspect ratio, and number of discrete voids. These results are visualized as void maps overlaid on the chip geometry, enabling engineers to assess the severity and location of filling defects. The void detection results provide critical feedback for process optimization, allowing adjustment of dispensing conditions (such as flow rate, dispensing location, or material viscosity), modification of bump layout or density to improve flow uniformity, or redesign of chip placement or gap height to minimize flow resistance and hesitation. By accurately predicting void formation, the simulation enables proactive identification of potential reliability issues and supports design-for-manufacturability efforts in advanced semiconductor packaging.

The method proceeds iteratively, allowing the user to modify input parameters and re-run the simulation to evaluate alternative designs or process conditions. The results from steps S810 and S812 can be stored, visualized, and exported for further analysis or integration with broader package design workflows.

Thus, the method provides a systematic and computationally efficient framework for hybrid simulation of capillary-driven underfill flow in complex chip assemblies. By integrating geometry import, zone classification, adaptive mesh generation, bump density modeling, multi-scale physics-based flow simulation, and automated void detection into a unified workflow, the method enables accurate prediction of underfill performance while maintaining practical computation times suitable for iterative design optimization and process development in high-volume semiconductor manufacturing environments.

The terminology used in the description is for describing particular embodiments only the terminology employed in the description of the various embodiments herein is intended for the purpose of describing particular embodiments and should not be construed as limiting. In the context of this description and the appended claims, the singular forms “a”, “an”, and “the” are intended to encompass plural forms as well, unless the context clearly indicates otherwise.

It should be understood that the term “and/or” as used herein is intended to encompass any and all possible combinations of one or more of the associated listed items. Furthermore, it should be noted that the terms “includes,” “including,” “comprises,” and/or “comprising,” when used in this specification, indicate the presence of stated features, integers, steps, operations, elements, and/or components, but do not exclude the presence or addition of one or more other features, integers, steps, operations, elements, components, and/or groups thereof.

Unless specifically stated otherwise, the term “some” refers to one or more. Various combinations using “at least one of” or “one or more of” followed by a list (e.g., A, B, or C) should be interpreted to include any combination of the listed items, including individual items and multiple items.

In the context of this disclosure, the terms “coupled,” “connected,” “connecting,” “electrically connected,” and similar expressions are used interchangeably to broadly denote the state of being electrically or electronically connected. Furthermore, an entity is deemed to be in “communication” with another entity (or entities) when it electrically transmits and/or receives information signals to/from the other entity, irrespective of whether these signals contain image/voice information or data/control information, and regardless of the signal type (analog or digital). It is important to note that this communication can occur through either wired or wireless means. The use of these terms is intended to encompass all forms of electrical or electronic connectivity relevant to the described embodiments.

The use of ordinal designators like “first,” “second,” and so forth in the specification and claims serves to differentiate between multiple instances of similarly named elements. These designators do not imply any inherent sequence, priority, or chronological order in the manufacturing process or functional relationship between elements. Rather, they are employed solely as a means of uniquely identifying and distinguishing between separate instances of elements that share a common name or description.

The directional terms used in the embodiments such as up, down, left, right, upper-side, down-side, in front of or behind are just the directions referring to the attached figures. Thus, the direction terms used in the present disclosure are for illustration, and are not intended to limit the scope of the present disclosure. It should be noted that the elements which are specifically described or labeled may exist in various forms for those skilled in the art.

As may be used throughout this specification and the appended claims, terms of approximation and degree such as “substantially,” “approximately,” “generally,” “essentially,” “nearly,” “about,” and similar expressions are used to account for variations in precision, manufacturing tolerances, measurement accuracy, environmental conditions, and inherent material properties that may affect the described features or characteristics. Such variations may range from ±20% in broader applications to progressively tighter tolerances of ±10%, ±5%, ±3%, ±2%, ±1%, or ±0.5% in more precise implementations. The specific degree of variation encompassed by these terms of approximation in any given context is informed by the nature of the component, relationship, or parameter being described, the technical requirements of the particular embodiment, and the understanding of one skilled in the relevant art.

The various illustrative components, logic, logical blocks, modules, circuits, operations and algorithm processes described in connection with the embodiments disclosed herein may be implemented as electronic hardware, firmware, software, or combinations of hardware, firmware or software, including the structures disclosed in this specification and the structural equivalents thereof. The interchangeability of hardware, firmware and software has been described generally, in terms of functionality, and illustrated in the various illustrative components, blocks, modules, circuits and processes described above. Whether such functionality is implemented in hardware, firmware or software depends upon the particular application and design constraints imposed on the overall system.

The hardware and data processing apparatus utilized to implement the various illustrative components, logics, logical blocks, modules, and circuits described herein may comprise, without limitation, one or more of the following: a general-purpose single-chip or multi-chip processor, a digital signal processor (DSP), an application specific integrated circuit (ASIC), a field programmable gate array (FPGA), other programmable logic devices (PLDs), discrete gate or transistor logic, discrete hardware components, or any suitable combination thereof. Such hardware and apparatus shall be configured to perform the functions described herein.

A general-purpose processor may include, but is not limited to, a microprocessor, or alternatively, any conventional processor, controller, microcontroller, or state machine. In certain implementations, a processor may be realized as a combination of computing devices. Such combinations may include, for example, a DSP and a microprocessor, a plurality of microprocessors, one or more microprocessors in conjunction with a DSP core, or any other such configuration as may be suitable for the intended application.

It is to be understood that in some embodiments, particular processes, operations, or methods may be executed by circuitry specifically designed for a given function. Such function-specific circuitry may be optimized to enhance performance, efficiency, or other relevant metrics for the particular task at hand. The selection of specific hardware implementation shall be determined based on the particular requirements of the application, which may include, inter alia, performance specifications, power consumption constraints, cost considerations, and size limitations.

In certain aspects, the subject matter described herein may be implemented as software. Specifically, various functions of the disclosed components, or steps of the methods, operations, processes, or algorithms described herein, may be realized as one or more modules within one or more computer programs. These computer programs may comprise non-transitory processor-executable or computer-executable instructions, encoded on one or more tangible processor-readable or computer-readable storage media. Such instructions are configured for execution by, or to control the operation of, data processing apparatus, including the components of the devices described herein. The aforementioned storage media may include, but are not limited to, Random Access Memory (RAM), Read Only Memory (ROM), Electrically Erasable Programmable Read-Only Memory (EEPROM), Compact Disc Read-Only Memory (CD-ROM) or other optical disk storage, magnetic disk storage or other magnetic storage devices, or any other medium capable of storing program code in the form of instructions or data structures. It should be understood that combinations of the above-mentioned storage media are also contemplated within the scope of computer-readable storage media for the purposes of this disclosure.

Some embodiments may involve computers on a distributed computing network, such as a network with multiple clients and/or servers. In such embodiments, clients may run software implementing client-side portions of the described systems and methods, while servers handle requests from these clients. Communication between clients and servers may occur via one or more electronic networks, which may include the Internet, wide area networks, mobile telephone networks, wireless networks (e.g., Wi-Fi, 5G), or local area networks, implemented using any known network protocols.

Various modifications to the embodiments described in this disclosure may be readily apparent to persons having ordinary skill in the art, and the generic principles defined herein may be applied to other embodiments without departing from the spirit or scope of this disclosure. Thus, the claims are not intended to be limited to the embodiments shown herein, but are to be accorded the widest scope consistent with this disclosure, the principles and the novel features disclosed herein.

In certain implementations, the embodiments may comprise the disclosed features and may optionally include additional features not explicitly described herein. Conversely, alternative implementations may be characterized by the substantial or complete absence of non-disclosed elements. For the avoidance of doubt, it should be understood that in some embodiments, non-disclosed elements may be intentionally omitted, either partially or entirely, without departing from the scope of the invention. Such omissions of non-disclosed elements shall not be construed as limiting the breadth of the claimed subject matter, provided that the explicitly disclosed features are present in the embodiment.

Additionally, various features that are described in this specification in the context of separate embodiments also can be implemented in combination in a single implementation. Conversely, various features that are described in the context of a single implementation also can be implemented in multiple embodiments separately or in any suitable subcombination. As such, although features may be described above as acting in particular combinations, and even initially claimed as such, one or more features from a claimed combination can in some cases be excised from the combination, and the claimed combination may be directed to a subcombination or variation of a subcombination.

The depiction of operations in a particular sequence in the drawings should not be construed as a requirement for strict adherence to that order in practice, nor should it imply that all illustrated operations must be performed to achieve the desired results. The schematic flow diagrams may represent example processes, but it should be understood that additional, unillustrated operations may be incorporated at various points within the depicted sequence. Such additional operations may occur before, after, simultaneously with, or between any of the illustrated operations.

Additionally, it should be understood that the various figures and component diagrams presented and discussed within this document are provided for illustrative purposes only and are not drawn to scale. These visual representations are intended to facilitate understanding of the described embodiments and should not be construed as precise technical drawings or limiting the scope of the invention to the specific arrangements depicted.

In certain implementations, multitasking and parallel processing may prove advantageous. Furthermore, while various system components are described as separate entities in some embodiments, this separation should not be interpreted as mandatory for all embodiments. It is contemplated that the described program components and systems may be integrated into a single software package or distributed across multiple software packages, as dictated by the specific implementation requirements.

It should be noted that other embodiments, beyond those explicitly described, fall within the scope of the appended claims. The actions specified in the claims may, in some instances, be performed in an order different from that in which they are presented, while still achieving the desired outcomes. This flexibility in execution order is an inherent aspect of the claimed processes and should be considered within the scope of the invention.

While the invention has been described in connection with certain embodiments, it will be understood by those skilled in the art that various modifications and adaptations can be made without departing from the scope of the invention. The specific embodiments presented are intended to illustrate the invention and not to limit its application or construction. Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system for simulating underfill flow during packaging of a chip assembly, comprising:
a memory storing instructions;
a processor coupled to the memory, and configured execute the instruction to:
import an assembly configuration of the chip assembly, a bump configuration of bumps on the chip assembly, and a dispensing condition of an underfill material to be introduced into the chip assembly;
define a simulation zone within the chip assembly based on the assembly configuration;
generate a simulation mesh corresponding to the simulation zone;
determine at least one bump group and a bump density of each bump group based on the bump configuration;
simulate flow of the underfill material into the simulation zone according to a thin plate model defined by the dispensing condition and the assembly configuration, and a porous media model defined by the at least one bump group and the bump density of each bump group; and
detect formation of voids in the underfill material based on the simulation.

2. The system of claim 1, wherein:
the simulation zone comprises a die-to-substrate area in the chip assembly;
the simulation mesh is a single layer 3D mesh describing the die-to-substrate area; and
the thin plate model employs a Hele-Shaw approximation to solve a drag force and a capillary force inflicted on the underfill flowing in the die-to-substrate area in a 2D field, thereby to simulate the underfill flowing in the die-to-substrate area in the single layer 3D mesh.

3. The system of claim 1, wherein:
the simulation zone comprises a die-to-die gap area in the chip assembly;
the simulation mesh is a vertically oriented 2D mesh describing the die-to-die gap area;
the thin plate model employs a Hele-Shaw approximation to solve a drag force, a capillary force and a hysteresis inflicted on the underfill flowing in the die-to-die gap in a 2D field; and
the hysteresis represents a corner effect of the underfill flowing from a die-to-substrate area to a die-to-die gap area.

4. The system of claim 1, wherein:
the simulation zone comprises a non-die area in the chip assembly;
the simulation mesh is a 3D mesh describing the non-die area; and
the processor is further configured to simulate the underfill flowing
in the non-die area using a Navier-Stokes equation to simulate the underfill flowing in the non-die area in the 3D mesh.

5. The system of claim 1, wherein:
the porous media model is used to compute a flow resistance inflicted on the underfill flowing into the simulation zone based on the bump density of the at least one bump group.

6. The system of claim 1, wherein:
the assembly configuration of the chip assembly comprises at least one property selected from a group consisting of: a 2D layout of die distribution on a substrate in the chip assembly, a material of each die in the chip assembly, a material of the substrate in the chip assembly, geometry of each die in the chip assembly, and a gap height between each die and the substrate in the chip assembly.

7. The system of claim 1, wherein:
the bump configuration of the bumps on the chip assembly comprises at least one property selected from a group consisting of: a 2D layout of bump distributions on a substrate in the chip assembly, a material of each bump in the chip assembly, geometry of each bump in the chip assembly.

8. The system of claim 1, wherein:
the dispensing condition comprises at least one property selected from a group consisting of: viscosity of the underfill, density of the underfill, a dispensing sequence of the underfill, dispensing time of the underfill, flow rate of the underfill, a contact angle between the underfill and solid material of the chip assembly.

9. A method for simulating underfill flow during packaging of a chip assembly, comprising:
importing an assembly configuration of the chip assembly, a bump configuration of bumps on the chip assembly, and a dispensing condition of an underfill material to be introduced into the chip assembly;
defining a simulation zone within the chip assembly based on the assembly configuration;
generating a simulation mesh corresponding to the simulation zone;
determining at least one bump group and a bump density of each bump group based on the bump configuration;
simulating flow of the underfill material into the simulation zone according to a thin plate model defined by the dispensing condition and the assembly configuration, and a porous media model defined by the at least one bump group and the bump density of each bump group; and
detecting formation of voids in the underfill material based on results of the simulation.

10. The method of claim 9, wherein:
the simulation zone comprises a die-to-substrate area in the chip assembly;
the simulation mesh is a single layer 3D mesh describing the die-to-substrate area; and
the simulating flow of the underfill material into the simulation zone according to a thin plate model comprises:
the thin plate model employing a Hele-Shaw approximation to solve a drag force and a capillary force inflicted on the underfill flowing in the die-to-substrate area in a 2D field, thereby to enable the dispensing simulation module to simulate the underfill flowing in the die-to-substrate area in the single layer 3D mesh.

11. The method of claim 9, wherein:
the simulation zone comprises a die-to-die gap area in the chip assembly;
the simulation mesh is a vertically oriented 2D mesh describing the die-to-die gap area;
the simulating flow of the underfill material into the simulation zone according to a thin plate model comprises:
the thin plate model employing a Hele-Shaw approximation to solve a drag force, a capillary force and a hysteresis inflicted on the underfill flowing in the die-to-die gap in a 2D field; and
the hysteresis represents a corner effect of the underfill flowing from a die-to-substrate area to a die-to-die gap area.

12. The method of claim 9, wherein:
the simulation zone comprises a non-die area in the chip assembly;
the simulation mesh is a 3D mesh describing the non-die area; and
the method further comprising employing a Navier-Stokes equation to simulate the underfill flowing in the non-die area in the 3D mesh.

13. The method of claim 9, wherein:
the simulating flow of the underfill material into the simulation zone according to a thin plate model comprises:
the porous media model computing a flow resistance inflicted on the underfill flowing into the simulation zone based on the bump density of the at least one bump group.

14. The method of claim 9, wherein:
the assembly configuration comprises at least one property selected from a group consisting of: a 2D layout of die distribution on a substrate in the chip assembly, a material of each die in the chip assembly, a material of the substrate in the chip assembly, geometry of each die in the chip assembly, and a gap height between each die and the substrate in the chip assembly.

15. The method of claim 9, wherein:
the bump configuration comprises at least one property selected from a group consisting of: a 2D layout of bump distributions on a substrate in the chip assembly, a material of each bump in the chip assembly, geometry of each bump in the chip assembly.

16. The method of claim 9, wherein:
the dispensing condition comprises at least one property selected from a group consisting of: viscosity of the underfill, density of the underfill, a dispensing sequence of the underfill, dispensing time of the underfill, flow rate of the underfill, a contact angle between the underfill and solid material of the chip assembly.
